(19) United States
(12) Patent Application Publication
(10) Pub. No.: US 2025/0138027 A1
(43) Pub. Date: May 1, 2025

(54) METHODS FOR IMPROVING LUNG TRANSPLANTS

(71) Applicant: Northwestern University, Evanston, IL (US)

(72) Inventor: Ankit BHARAT, Evanston, IL (US)

(73) Assignee: Northwestern University, Evanston, IL (US)

(21) Appl. No.: 18/932,109

(22) Filed: Oct. 30, 2024

Related U.S. Application Data
(60) Provisional application No. 63/594,499, filed on Oct. 31, 2023.

(51) Int. Cl. G01N 33/68

(52) U.S. Cl. CPC G01N 33/6893; G01N 2333/4716; G01N 2800/245; G01N 2800/50

(57) ABSTRACT

Provided herein are methods to predict primary graft dysfunction in a subject, comprising detecting and/or quantifying complement protein in allograft biopsy tissue up to 120 minutes post-reperfusion.

### TECHNICAL FIELD

This application claims the benefit under 35 U.S.C. § 119(e) of U.S. Provisional Application Ser. No. 63/594,499, filed Oct. 31, 2023, the contents of which are hereby incorporated by reference in its entirety.

### STATEMENT OF GOVERNMENT SUPPORT

This invention was made with government support under grant number HL145478 awarded by the National Institutes of Health. The government has certain rights in the invention.

### TECHNICAL FIELD

The present invention relates to methods for improving organ transplants.

### BACKGROUND

Primary Graft Dysfunction (PGD) is a dreaded complication after solid organ transplants.^{1, 54} It is particularly high post-lung transplant, manifesting within the initial 72 hours.¹ PGD occurs within 24 hours after heart transplantation.⁵⁴ Characterized by diffuse pulmonary edema and hypoxemia with no identifiable alternative cause, PGD holds significant correlation with both short and long-term mortality following lung and heart transplantation. It is also correlated with chronic lung allograft rejection. Despite extensive studies identifying risk factors, an absence of early predictors and targeted therapies perpetuates a substantial incidence of over 50% (lungs) and 3-30% (heart) for PGD.^{2-7, 54}

The primary trigger of PGD post organ transplant is ischemia-reperfusion injury.⁸⁻¹² For example, non-classical monocytes, retained within donor lungs, serve as the sentinel cells that recruit neutrophils, initiating PGD.¹³ Simultaneously, these monocytes activate donor alveolar macrophages, culminating in the release of MCP-1.¹⁴ This mobilizes classical monocytes from the recipient spleen, leading to the permeabilization of the pulmonary vasculature through the release of IL1b—a vital step for neutrophil extravasation and the initiation of NETosis, driving ischemia-reperfusion injury.¹⁵ The neutrophils can, however, also activate complement cascade during this process.¹⁶

Complement activation has been implicated as a mediator in the pathogenesis of PGD following lung transplantation in both animal models and human studies.^{11, 40 19-21, 41-46} The relevance of complement activation in the pathogenesis of PGD is further affirmed by reports indicating a rise in plasma C5a within 6-24 hours post-transplant correlated with severe grades of PGD.^{17, 19-22} Additional previous studies have associated PGD with elevated post-operative plasma C5a levels, bronchoalveolar lavage fluid complement activation fragments, and positive C3d and C4d staining on transbronchial lung biopsies.^{12, 19, 20, 38} Further, in serum samples collected prior to transplant, the complement system was triggered in heart transplant patients who later developed PGD.⁵⁵

Complement activation certainly plays a role in the pathogenesis lung allograft dysfunction mediated by lung-restricted autoantibodies, which is increasingly recognized as an etiology for a subset of PGD cases.^{8-12, 17} However, whether complement activation also plays a causal role in ischemia-reperfusion injury, the primary cause of PGD post-lung transplantation, in the absence of HLA (human leukocyte antigen) and lung-restricted autoantibodies remains unclear. In a previous study, complement activation following allogeneic or syngeneic murine lung transplantation was not observed when using physiologic limits of ischemia-reperfusion.¹⁷ It is possible that poor organ preservation or tissue damage can activate tissue necroptosis⁴⁷ and consequently the alternate pathway of complement, independent of antibodies.

While multiple studies have identified risk factors for PGD following lung transplantation, predicting those patients who will develop significant PGD remains a challenge as early, reliable clinical markers are lacking.^{2, 3, 7, 24} Recent studies have uncovered the ability of post-reperfusion extravascular lung water and systolic pulmonary artery pressure to predict PGD.^{29, 48}

Thus, there exists an unmet need for early biomarkers of PGD.

### SUMMARY OF THE INVENTION

One embodiment is a method for predicting primary graft dysfunction in a subject comprising quantifying a complement protein in an allograft biopsy, wherein the allograft biopsy is obtained from an organ up to 120 minutes post-reperfusion following the transplant of the organ in the subject, wherein a level of the complement protein in the allograft biopsy that is higher than a quantity of the same complement protein in a biopsy from the same organ at the end of cold ischemia indicates a likelihood of primary graft dysfunction. Particularly, in some embodiments the organ is a lung.

Another embodiment is a method for predicting primary graft dysfunction in a subject comprising detecting a complement protein in an allograft biopsy, wherein the allograft biopsy is obtained from an organ up to 120 minutes post-reperfusion following the transplant of the organ in the subject. Particularly, in some embodiments the organ is a lung.

The detection and quantification of complement protein post-reperfusion provides an early indicator of primary graft dysfunction in a subject.

Without wishing to be bound by theory, in the absence of any directed treatment of PGD currently, Applicant postulates that the finding of complement deposition early following reperfusion is significant since it introduces identification of patients who might have a mitigable risk of severe PGD through the use of complement inhibition which have shown promising results.^{17, 21, 22} Furthermore, plasmapheresis is an emerging treatment for ischemia-reperfusion injury and PGD in other solid organ transplants and has been noted to mitigate PGD in a subset of lung transplant patients.^{17, 49-51}

Since complement can be activated within minutes of the inciting injury, without wishing to be bound by theory Applicant hypothesized that detecting complement activation in post-reperfusion allograft biopsies obtained during the lung transplant procedure could identify patients at risk for severe PGD at 72 hours which is known to most strongly correlate with poor transplant outcomes.²³ Further, this hypothesis could extend to identifying patients at risk for severe PGD after other solid organ transplantation.

### BRIEF DESCRIPTION OF THE DRAWINGS

FIG. 1 shows a lung transplant allograft biopsy with no C4d deposition visible in capillaries and small vessels.

FIG. 2 shows a lung transplant allograft biopsy with positive C4d staining. Note crisp lining of C4d deposition along the endothelial cells of capillaries and small vessels.

FIG. 3 shows a survival curve comparing patients with PGD grade 0-2 and PGD grade 3. At one-year post-transplant, overall survival was significantly reduced in those patients with PGD grade 3 compared to those with PGD grade 0-2 (65.1% vs. 90.5%, p<0.001).

FIGS. 4A-4B show (FIG. 4A) Bronchoalveolar lavage fluid levels of soluble complement proteins from new allograft between 2-4 hours following reperfusion after chest closure by C4d staining grade (p<0.01). (FIG. 4B) Bronchoalveolar lavage fluid level of soluble C4d from allograft vs. native lung in single lung transplant recipients who had grade 3 C4d staining at 30 minutes post-reperfusion (* p<0.001).

FIG. 5 shows a survival curve comparing patients with positive and negative post-reperfusion lung transplant biopsy C4d staining. At one-year post-transplant, overall survival was significantly reduced in those patients with positive C4d staining compared to those with negative C4d staining (80.4% vs. 88.2%, p=0.02).

### DETAILED DESCRIPTION

### Definitions

Unless defined otherwise, all technical and scientific terms used herein generally have the same meaning as commonly understood by one of ordinary skill in the art to which this technology belongs.

Unless otherwise specified, “a” or “an” means “one or more” unless clearly specified otherwise.

The term “subject” as used herein refers to living multi-cellular organisms, including vertebrate organisms, a category that include both human and non-human mammals. The methods and compositions as disclosed herein have equal application in medical and veterinary settings. Thus, the general term “subject” under the treatment is understood to include all animals, such as humans, domestic animals, wild animals, and laboratory animals. “Subject” and “patient” may be used interchangeably, unless otherwise noted.

The term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “protein,” “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively.

### Modes of Carrying Out the Disclosure

### Methods to Predict Primary Graft Dysfunction

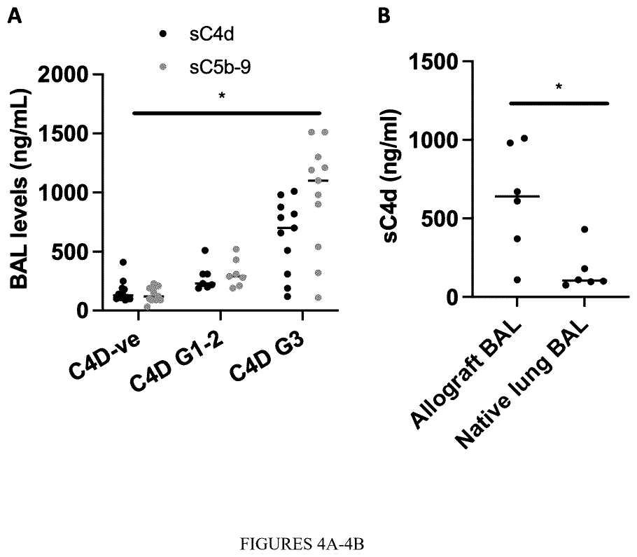

Contemplated herein are methods for predicting primary graft dysfunction in a subject associated with the presence of complement proteins. In some embodiments, this includes PGD prediction in a subject who underwent a lung transplant. In some embodiments, this includes PGD prediction in a subject who underwent a heart transplant. In some embodiments, this includes identifying a subject with severe (level 3) PGD (see Table 4, FIG. 4A). In other embodiments, this includes identifying a subject with mild (levels 1 or 2) PGD. PGD severity is determined based on the 2016 consensus statement of the International Society for Heart and Lung Transplantation (“ISHLT”)¹: PGD grade 0 (no pulmonary edema on chest X-ray, any PaO₂/FiO₂ ratio), PGD grade 1 (pulmonary edema on chest X-ray, PaO₂/FiO₂ ratio >300), PGD grade 2 (pulmonary edema on chest X-ray, PaO₂/FiO₂ ratio 200-300), PGD grade 3 (pulmonary edema on chest X-ray, PaO₂/FiO₂ ratio <200), where “PaO₂” is the partial pressure of arterial oxygen and “FiO₂” is the fraction of inspired oxygen. According to the ISHLT, the oxygen saturation/FiO₂ ratio may be used, with the grading values shifted accordingly, if PaO₂ is not available.¹

More particularly, contemplated herein are methods for predicting primary graft dysfunction in a subject through detection and/or quantification of complement proteins in allograft biopsy tissue. The term “quantified” as used herein can refer to exact quantities of a measured substance or can refer to semi-quantitative values of a measured substance. For example, complement proteins quantified in an allograft biopsy may be scored on a semi-quantitative scale of 0 to 3. The term “detected” as used herein refers to the presence of a measured substance. In some embodiments, detection of a complement protein is done with immunohistochemical staining or immunofluorescence.

The term “subject” includes mammalian animals (mammals), such as a non-human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models known in the art.

Complement is the collective term for a series of blood proteins and is a major effector mechanism of the immune system. Complement activation and its deposition on target structures can lead to direct complement-mediated cell lysis or can lead indirectly to cell or tissue destruction due to the generation of powerful modulators of inflammation and the recruitment and activation of immune effector cells. Complement activation products that mediate tissue injury are generated at various points in the complement pathway. Inappropriate complement activation on host tissue plays an important role in the pathology of many autoimmune and inflammatory diseases, and is also responsible for many disease states associated with bioincompatibility, e.g. post-cardiopulmonary inflammation and transplant rejection. Complement deposition on host cell membranes is prevented by complement inhibitory proteins expressed at the cell surface.

The complement system comprises a collection of about 30 proteins and is one of the major effector mechanisms of the immune system. The complement cascade is activated principally via either the classical (usually antibody-dependent) or alternative (usually antibody-independent) pathways. Activation via either pathway leads to the generation of C3 convertase, which is the central enzymatic complex of the cascade. C3 convertase cleaves serum C3 into C3a and C3b, the latter of which binds covalently to the site of activation and leads to the further generation of C3 convertase (amplification loop). The activation product C3b (and also C4b generated only via the classical pathway) and its breakdown products are important opsonins and are involved in promoting cell-mediated lysis of target cells (by phagocytes and NK cells) as well as immune complex transport and solubilization. C3/C4 activation products and their receptors on various cells of the immune system are also important in modulating the cellular immune response. C3 convertases participate in the formation of C5 convertase, a complex that cleaves C5 to yield C5a and C5b. C5a has powerful proinflammatory and chemotactic properties and can recruit and activate immune effector cells. Formation of C5b initiates the terminal complement pathway resulting in the sequential assembly of complement proteins C6, C7, C8 and (C9) n to form the membrane attack complex (MAC or C5b-9). Formation of MAC in a target cell membrane can result in direct cell lysis, but can also cause cell activation and the expression/release of various inflammatory modulators. Information about the structure of MAC is found in Bayly-Jones et al. (2017) Philos Trans R Soc Lond B Biol Sci. 5:372 (doi: 10.1098/rstb.2016.0221), which is incorporated herein in its entirety.

C3d is a degradation product of C3. Additional information about complement C3d is found in “Complement C3d” [MeSH Terms] at ncbi.nlm.nih.gov/mesh?Db=mesh&Cmd=DetailsSearch&Term=%22Complement+C3d%22%5BMeSH+Terms%5D (last accessed Oct. 27, 2024), which are incorporated herein by reference in its entirety. A non-limiting exemplary sequence of C3, including C3d, may be found under UniProtKB P01024, which is incorporated herein in its entirety.

C4d is a degradation product of C4. Human C4 may have isotypes C4A and C4B. See Li et al 2017 Scientific Reports 7:42628 (doi: 10.1038/srep42628), which is incorporated herein in its entirety. A non-limiting exemplary sequence of isoform C4-A, including C4d-A, may be found under UniProtKB P0C0L4, which is incorporated herein in its entirety. A non-limiting exemplary sequence of isoform C4-B, including C4d-B, may be found under UniProtKB P0C0L5.

As used in the methods described herein to predict PGD and treat PGD, the term “complement protein” includes complement proteins, complement protein complexes and complement protein fragments and complement protein degradation products.

In one embodiment to predict PGD, the method comprises, consists of, or consists essentially of quantifying a complement protein in an allograft biopsy taken from an organ up to 120 minutes post-reperfusion and comparing with the same complement protein quantified in a biopsy taken at the end of cold ischemia from the same organ, and PGD is predicted if the post-reperfusion quantity of complement protein is higher.

In another embodiment to predict PGD, the method comprises, consists of, or consists essentially of detecting a complement protein in an allograft biopsy taken from an organ up to 120 minutes post-reperfusion.

More particularly, in one embodiment to predict PGD, the method comprises, consists of, or consists essentially of quantifying a complement protein in an allograft biopsy taken from a lung up to 120 minutes post-reperfusion and comparing the quantity with the same complement protein quantified in a biopsy taken at the end of cold ischemia from the same lung, and PGD is predicted if the post-reperfusion quantity of the complement protein is higher. The biopsy taken at the end of cold ischemia is the internal control.

In another embodiment to predict PGD, the method comprises, consists of, or consists essentially of detecting a complement protein in an allograft biopsy obtained from a lung up to 120 minutes post-reperfusion.

In another embodiment to predict PGD, the method comprises, consists of, or consists essentially of quantifying the complement protein in an allograft biopsy taken from a heart up to 120 minutes post-reperfusion and comparing with the complement protein quantified in a biopsy taken at the end of cold ischemia from the heart, and PGD is predicted if the post-reperfusion quantity of the same complement protein is higher. The biopsy taken at the end of cold ischemia is the internal control.

In another embodiment to predict PGD, the method comprises, consists of, or consists essentially of detecting a complement protein is detected in an allograft biopsy obtained from a heart up to 120 minutes post-reperfusion.

Preferably, PGD is predicted if the post-reperfusion quantity of complement protein, is at least 1.2-fold greater in the allograft biopsy when compared to the same complement protein quantified in the biopsy taken from the same organ at the end of cold ischemia. In some embodiments, the complement protein is not at quantifiable levels in the biopsy from the organ at the end of cold ischemia.

In some aspects, the allograft biopsy is obtained 30 minutes post-reperfusion. In some embodiments, the allograft biopsy is obtained up to 90 minutes post-reperfusion. In some embodiments the allograft biopsy is obtained between 30 and 120 minutes post-reperfusion.

In yet another embodiment to predict PGD, complement protein is detected in a transbronchial biopsy, where the transbronchial biopsy is obtained from a lung up to 72 hours post-transplant of the lung in the subject.

In some embodiments the complement protein is quantified and/or detected using immunohistochemical staining. In other embodiments the complement protein is quantified and/or detected using immunofluorescence.

In one aspect, the biopsies are wedge biopsies.

In some embodiments, the complement protein is selected from a group comprising C4d, C5b-9, and C3d. In one aspect, the complement protein is C4d (see e.g. Tables 5 and 6). In other embodiments, the complement protein is selected from a different complement protein.

In some embodiments, the quantity or presence of complement protein in the allograft biopsy can further be used to predict post-transplant outcomes. In some aspects, the post-transplant outcomes are selected from the group comprising digital ischemia, post-transplant Extracorporeal Membrane Oxygenation (ECMO) support, and a need for dialysis (see Table 6).

### Methods for Treating Primary Graft Dysfunction

Also contemplated herein are methods for treating primary graft dysfunction in a subject, the method comprising detecting a complement protein in an allograft biopsy, wherein the allograft biopsy is obtained from an organ up to 120 minutes post-reperfusion following the transplant of the organ in the subject, and treating the subject with a treatment for primary graft dysfunction when the complement protein level is at least 1.2-fold higher than a quantity of the same complement protein in a biopsy from the same organ at the end of cold ischemia. In some embodiments, the treatment for primary graft dysfunction is complement inhibition therapy.

The following example further illustrates though in no way limits the scope of the foregoing embodiments.

### Example 1

### Temporal Correlation Between Post-Reperfusion Complement Deposition and Severe Primary Graft Dysfunction in Lung Allografts

This study was conducted to assess early complement activation post-reperfusion in lung transplant patients to determine whether early complement activation could serve as a biomarker for severe primary graft dysfunction (PDG-3). This study provided evidence that early complement deposition in allograft tissue is highly predictive of PGD-3 at 72 hours post-transplant.

### Summary

Consecutive lung transplant patients (n=253) from January 2018 through June 2023 underwent timed open allograft biopsies at the end of cold ischemia (internal control) and 30 minutes post-reperfusion. PGD-3 at 72 hours occurred in 14% (35/253) of patients, 17% (44/253) revealed positive C4d staining on post-reperfusion allograft biopsy, and no biopsy related complications were encountered. Significantly more patients with PGD-3 at 72 hours had positive C4d staining at 30 minutes post-reperfusion compared to those without (51% vs 12%, p<0.001). Conversely, patients with positive C4d staining were significantly more likely to develop PGD-3 at 72 hours (41% vs 8%, p<0.001) and experienced worse long-term outcomes. In multivariate logistic regression, positive C4d staining remained highly predictive of PGD-3 (OR 7.92, 95% CI 2.97-21.1, p<0.001). Hence, early complement deposition in allograft is highly predictive of PGD-3 at 72 hours.

### Methods and Patients

Study Design: This study was approved by the Institutional Review Board (STU00207250 and STU00213616) and they approved waiver of consent. This is a retrospective review of 309 patients who underwent lung transplant at a single institution from January 2018 through June 2023. Those undergoing re- or multi-organ transplant, and those without C4d staining were excluded (n=56) and the final cohort included 253 patients. Sample size calculations showed 35 patients with PGD grade 3 were necessary for an effect size to obtain a 90% confidence level with 10% margin of error, assuming 15% of lung transplants develop PGD grade 3.^{6, 24} Data collected included patients' and donor characteristics and intra- and post-operative outcomes. Recipient characteristics, e.g. lab values, were collected just prior to lung transplant. Standardized criteria donor assessment and procurements as proposed by the Professional practice consensus guidelines of the International Society of Heart and Lung Transplantation are used.²⁵ Recipients typically receive pre-operative induction therapy consisting of solumedrol and basiliximab followed by maintenance therapy with a calcineurin inhibitor, antimetabolite, and steroids.

C4d Staining: Study patients underwent a sequential 1×1 cm biopsy from the same region of the upper lobe at the end of cold ischemia (internal control) and at 30 minutes post-reperfusion from the first lung implanted. In some cases, additional biopsies were obtained in situ in the donor, 5 minutes and 90/120 minutes following reperfusion. Each serial biopsy incorporated the prior staple line and, therefore, at the end of the biopsy there was only one staple line remaining in the patients at the site of biopsy. Early feasibility studies revealed 30 minutes to have a 60% higher yield for complement deposition compared to 5 minutes but no difference between 30 minutes and longer time points. Hence, for the current study, the second biopsy was obtained at 30 minutes to not interfere with chest closure or prolong operative time.

Post-transplant biopsies were also taken periodically in patients post-transplant to determine the survival rate in Cd4 negative and positive patients.

Tissue sections from the biopsies were fixed in 10% buffered formalin and embedded in paraffin. For routine microscopy, 4-μm-thick sections were stained with hematoxylin-eosin.

Immunohistochemical staining was performed using an automated immunostainer (Ventana BanchMark ULTRA; Ventana Medical Systems, Inc, Tucson, AZ), rabbit prediluted C4d monoclonal antibody (Clone SP91, Cell Marque, Rocklin, CA) and ultra View Universal biotin-free DAB Detection Kit.

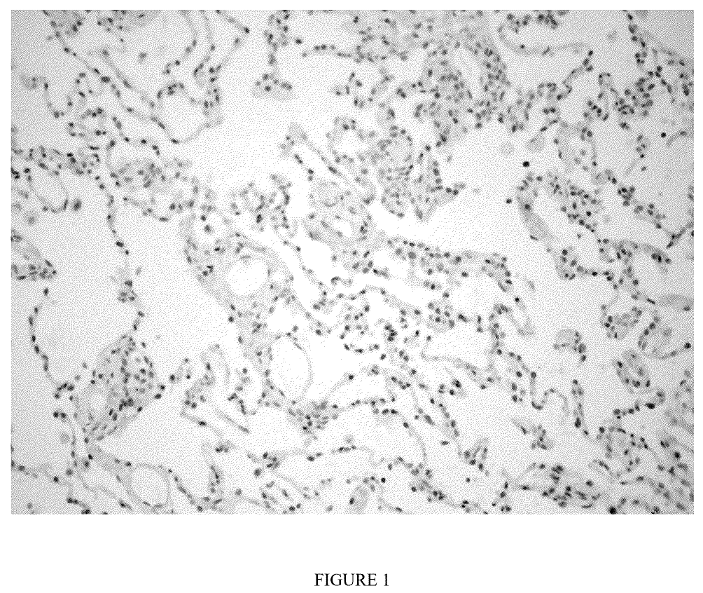

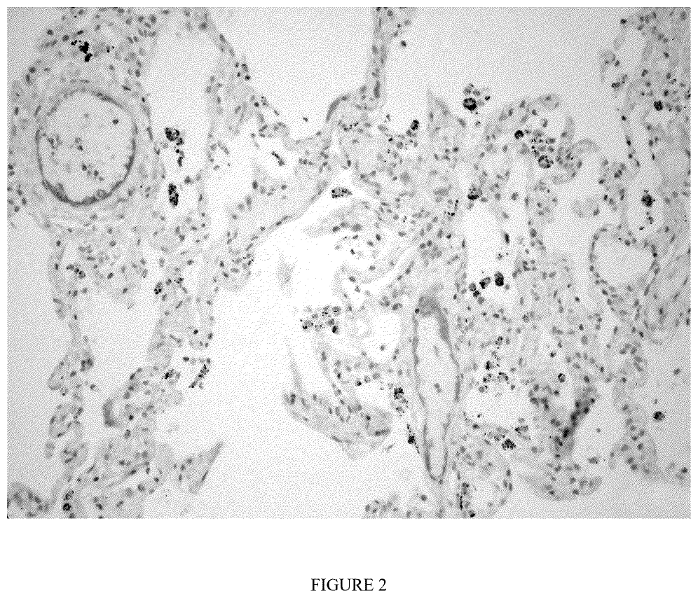

Continuous, linear, endothelial/sub-endothelial staining of C4d in capillaries was considered positive, as recommended by the 2007 International Society for Heart and Lung Transplantation (ISHLT) guidelines.²⁶ C4d scoring was semi-quantitative and recorded as 0 (no staining) (FIG. 1), 1+ (<10% of capillaries), 2+ (10% to 50%) or 3+ (>50%), similar to the ISHLT working formulation for antibody-mediated rejection in heart transplantation.²⁷ Two lung transplant pathologists independently scored all C4d-stained slides and following reached consensus regarding the final result. Both pathologists were blinded to the PGD status. Post-reperfusion specimens with a score of 1+, 2+ or 3+ that were increased from the pre-reperfusion biopsy samples were considered C4d positive (FIG. 2).

PGD: PGD was defined and graded based on the ISHLT guidelines.^{1, 23} If PaO₂ was unavailable for calculation of the PaO₂/FiO₂ ratio, then an oxygen saturation/FiO₂ ratio was used. The PaO₂/FiO₂ ratio at 72 hours after lung transplantation was used for determining PGD grade in both single and bilateral lung transplants. The use of Extracorporeal Membrane Oxygenation (ECMO) with bilateral pulmonary edema on C×R images was considered grade 3.

In a separate cohort, complement proteins were tested in the bronchoalveolar lavage fluid using soluble C5b-9 assay (BD OptEIA Human C5b-9 ELISA set) and the soluble C4d assay (Quidel Micro Vue ELISA), similar to the methods of Kulkarni et al. (2020) who quantified complement protein in bronchoalveolar lavage fluid at approximately two hours after allograft reperfusion.

Statistical Analysis: Continuous variables were analyzed using t-tests and presented as means±standard deviation, while categorical variables were compared using Fisher's exact tests and reported as number (percentage). P-values <0.05 were accepted as statistically significant. Univariate logistic regression analyses were utilized to assess the ability of recipient and donor characteristics, C4d staining, and other intra-operative outcomes to predict PGD grade 3. Following univariate analysis, a multivariate logistic regression analysis was performed using selected variables from the univariate logistic regression analyses with a p-value <0.05.^{17, 18, 28-32}

Comparative analysis was also done between recipient and donor characteristics, as well as intra- and post-operative outcomes, and between patients with positive and negative post-reperfusion lung transplant biopsy C4d staining.

All analyses were performed using EZR software (Saitama Medical Center, Jichi Medical University, Saitama, Japan), a graphical user interface for R (The R Foundation for Statistical Computing, Vienna, Austria).

### Results

Study Cohort: In total, 253 patients met study inclusion criteria. Of these, 104/253 (41.1%) were female and 163/253 (64.4%) underwent bilateral lung transplantation. The average age of patients was 58 years, with an average Body Mass Index of 26.0 kg/m² and pre-CAS average Lung Allocation Score of 55.9 (Table 1). No patients in the study died within the first 72 hours post-transplant without re-transplant or without a diagnosis of PGD, and no patients developed prolonged air leak (>5 days), hemothorax, or prolonged hospitalization attributable to the wedge biopsies performed.

Patient Characteristics associated with PGD grade 3: There were 218 (86.2%) patients who developed PGD grade 0-2 and 35 (13.8%) who developed PGD grade 3. Patients who developed PGD grade 3 were significantly more likely to be younger (53.1±12.6 vs. 59.1±12.2 years, p<0.01), have COVID-19 ARDS lung failure (11 (31.4%) vs. 29 (13.3%), p=0.01), require pre-transplant ECMO support (15 (42.9%) vs 17 (7.8%), p<0.001), and have higher lung allocation scores (68.6±22.4 vs. 53.9±18.0, p<0.001) than those who developed PGD grade 0-2. Additionally, patients who developed PGD grade 3 had significantly lower pre-operative hemoglobin (10.2±3.3 vs. 11.7±2.5 g/dL, p<0.01) and albumin (3.6±0.7 vs. 4.0±0.5 g/dL, p<0.001), and significantly higher pre-operative total bilirubin (1.0±1.1 vs. 0.6±0.4 mg/dL, p<0.001) and INR (1.2±0.4 vs. 1.1±0.2, p<0.01). There were no significant differences in donor characteristics between the two cohorts (Table 1).

TABLE 1
Patient characteristics by PGD grade.

| | PGD grade 0-2 | | PGD grade 3 | | |
|---|---|---|---|---|---|
| Variable | (n = 218) | | (n = 35) | | P value |
| Recipient factors | | | | | |
| Age, years | 59.1 ± 12.2 | | 53.1 ± 12.6 | | <0.01 |
| Female | 87 | (39.9%) | 17 | (48.6%) | 0.36 |
| Body Mass Index, kg/m² | 26.1 ± 4.6 | | 25.5 ± 4.5 | | 0.51 |
| Body Surface Area, m² | 1.9 ± 0.2 | | 1.8 ± 0.2 | | 0.47 |
| Smoking history | 106 | (48.6%) | 14 | (40.0%) | 0.37 |
| Hypertension | 114 | (52.3%) | 15 | (42.9%) | 0.36 |
| Diabetes | 69 | (31.7%) | 11 | (31.4%) | 1.00 |
| Chronic Kidney Disease | 12 | (5.5%) | 5 | (14.3%) | 0.07 |
| Pre-transplant ECMO use | 17 | (7.8%) | 15 | (42.9%) | <0.001 |
| Bilateral lung transplant | 137 | (62.8%) | 26 | (74.3%) | 0.25 |
| Lung Allocation Score | 53.9 ± 18.0 | | 68.6 ± 22.4 | | <0.001 |
| Composite Allocation Score | 26.6 ± 9.2 | | 27.6 ± 9.0 | | 0.84 |
| On the waiting list | 14 | [6-41] | 15 | [5-39] | 0.90 |
| Etiology of lung failure | | | | | |
| Interstitial Lung Disease | 85 | (39.0%) | 12 | (34.3%) | 0.71 |
| COVID-19 | 29 | (13.3%) | 11 | (31.4%) | 0.01 |
| COPD | 41 | (18.8%) | 3 | (8.6%) | 0.16 |
| Pulmonary Artery | 20 | (9.2%) | 2 | (5.7%) | 0.75 |
| Hypertension | | | | | |
| Other | 43 | (19.7%) | 7 | (20.0%) | 1.00 |
| Laboratory values | | | | | |
| Hemoglobin, g/dL | 11.7 ± 2.5 | | 10.2 ± 3.3 | | <0.01 |
| WBC, 1,000/mm³ | 9.7 ± 3.7 | | 9.8 ± 3.7 | | 0.81 |
| Platelets, 1,000/mm³ | 252.8 ± 91.0 | | 220.1 ± 91.6 | | 0.05 |
| Sodium, mEq/L | 139.6 ± 3.5 | | 140.6 ± 3.9 | | 0.10 |
| BUN, mg/dL | 16.0 ± 6.9 | | 18.5 ± 9.5 | | 0.07 |
| Creatinine, mg/dL | 0.78 ± 0.25 | | 0.82 ± 0.29 | | 0.46 |
| AST, U/L | 25.5 ± 19.3 | | 31.9 ± 24.8 | | 0.08 |
| ALT, U/L | 21.1 ± 18.1 | | 22.0 ± 15.8 | | 0.78 |
| Albumin, g/dL | 4.0 ± 0.5 | | 3.6 ± 0.7 | | <0.001 |
| Total bilirubin, mg/dL | 0.6 ± 0.4 | | 1.0 ± 1.1 | | <0.001 |
| INR | 1.1 ± 0.2 | | 1.2 ± 0.4 | | <0.01 |
| PRA* | 83 | (38.1%) | 19 | (54.3%) | 0.09 |
| Arterial Blood Gas | | | | | |
| pH | 7.38 ± 0.07 | | 7.37 ± 0.08 | | 0.80 |
| PaCO₂ | 49.2 ± 10.8 | | 48.5 ± 13.4 | | 0.74 |
| PaO₂ | 271.9 ± 111.1 | | 231.3 ± 111.7 | | 0.05 |
| Donor | | | | | |
| Age, years | 33.4 ± 12.0 | | 32.9 ± 11.6 | | 0.81 |
| Female | 57 | (26.1%) | 14 | (40.0%) | 0.11 |
| Cause of death | | | | | |
| Anoxia | 85 | (39.0%) | 13 | (37.1%) | 1.00 |
| Head trauma | 82 | (37.6%) | 14 | (40.0%) | 0.85 |
| Other | 51 | (23.4%) | 8 | (22.9%) | 1.00 |

Continuous data are shown as means ± standard deviation (SD) for age and laboratory data, and as medians and interquartile ranges [Q1-Q3] for days.
ALT, Alanine aminotransferase; ARDS, acute respiratory distress syndrome; AST, aspartate aminotransferase; BUN, blood urea nitrogen; COPD, chronic obstructive pulmonary disease; COVID-19, coronavirus disease 2019; ECMO; extracorporeal membrane oxygenation; INR, international normalized ratio; PGD, primary graft dysfunction; PRA, panel reactive antibodies; WBC, white blood cell.
Etiology of lung failure other: sarcoidosis, Hypersensitivity Pneumonitis, Cystic Fibrosis, Bronchiectasis, Obliterative bronchiolitis, Bronchoalveolar Carcinoma, Primary Ciliary Dyskinesia.
*PRA described as a count as this is an indicator of any sensitization (i.e. PRA > 0).

Outcomes associated with PGD grade 3: Intra-operatively, patients who developed PGD grade 3 had significantly longer operative times (8.2 [5.5-9.3] vs. 6.0 [5.0-7.8] hours, p<0.001) and more blood product transfusions, including packed Red Blood Cells (pRBC) (3 [1-11] vs. 1 [0-2] units, p<0.01), Fresh Frozen Plasma (FFP) (0 [0-4] vs. 0 [0-0] units, p<0.001), and platelets (0 [0-3] vs. 0 [0-0] units, p<0.001) than those patients who developed PGD grade 0-2. Additionally, recipients with PGD grade 3 had a notably higher incidence of positive post-reperfusion lung transplant biopsy C4d staining (18 (51.4%) vs. 26 (11.9%), p<0.001), and, specifically, C4d staining grade 3 (5 (27.7%) vs. 1 (3.8%), p=0.02).

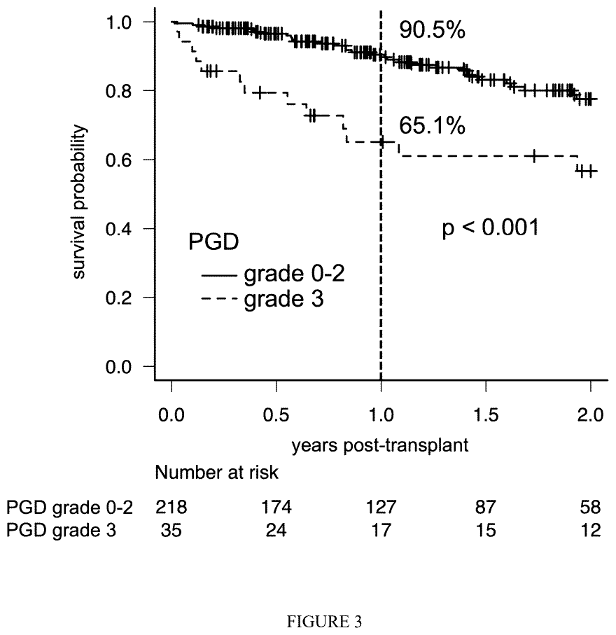

Post-operatively, patients who developed PGD grade 3 were more likely to require ECMO (25 (71.4%) vs. 12 (5.5%), p<0.001), and had longer intensive care unit (ICU) admissions (21 [11-38] vs. 7 [5-15] days, p<0.001), post-transplant ventilator requirements (9 [2-18] vs. 2 [1-3] days, p<0.001), and overall hospital stays (35 [18-50] vs. 17 [12-27] days, p<0.001). Patients with PGD grade 3 also had significantly higher incidences of post-operative Acute Kidney Injury (AKI) (28 (80.0%) vs. 84 (38.5%), p<0.001), dialysis use (22 (62.9%) vs. 14 (6.4%), p<0.001), bowel ischemia (3 (8.6%) vs. 1 (0.5%), p<0.01), and digital ischemia (4 (11.4%) vs. 0 (0%), p<0.001) (Table 2). Furthermore, one year survival was significantly reduced in those recipients who developed PGD grade 3 compared to those who developed PGD grade 0-2 (FIG. 3).

TABLE 2
Intra- and post-operative outcomes of lung transplant recipients by PGD.

| | PGD grade 0-2 | | PGD grade 3 | | |
|---|---|---|---|---|---|
| Variable | (n = 218) | | (n = 35) | | P value |
| Intra-operative outcomes | | | | | |
| Lung transplant biopsy C4d | 26 | (11.9%) | 18 | (51.4%) | <0.001 |
| staining | | | | | |
| C4d staining grade 1 | 14 | (53.8%) | 5 | (27.7%) | 0.08 |
| C4d staining grade 2 | 11 | (42.3%) | 8 | (44.4%) | 0.88 |
| C4d staining grade 3 | 1 | (3.8%) | 5 | (27.7%) | 0.02 |
| Operative time (hours) | 6.0 | [5.0-7.8] | 8.2 | [5.5-9.3] | <0.001 |
| Intra-op blood transfusion; pRBC | 1 | [0-2] | 3 | [1-11] | <0.001 |
| Intra-op blood transfusion; FFP | 0 | [0-0] | 0 | [0-4] | <0.001 |
| Intra-op blood transfusion; Plt | 0 | [0-0] | 0 | [0-3] | <0.001 |
| Ischemic time (hours) | 5.2 | [4.2-5.9] | 5.2 | [4.2-5.8] | 0.78 |
| Veno-arterial ECMO use | 145 | (66.5%) | 27 | (77.1%) | 0.25 |
| Veno-arterial ECMO time (hours) | 2.4 | [0-3.1] | 2.7 | [0-3.3] | 0.39 |
| Post-operative outcomes | | | | | |
| Acute Kidney Injury | 84 | (38.5%) | 28 | (80.0%) | <0.001 |
| Dialysis | 14 | (6.4%) | 22 | (62.9%) | <0.001 |
| Stroke | 3 | (1.4%) | 2 | (5.7%) | 0.14 |
| Bowel ischemia | 1 | (0.5%) | 3 | (8.6%) | <0.01 |
| Digital ischemia | 0 | (0%) | 4 | (11.4%) | <0.001 |
| Post-transplant ECMO support | 12 | (5.5%) | 25 | (71.4%) | <0.001 |
| Intensive Care Unit stay (days) | 7 | [5-15] | 21 | [11-38] | <0.001 |
| Post-transplant ventilator (days) | 2 | [1-3] | 9 | [2-18] | <0.001 |
| Hospital stay (days) | 17 | [12-27] | 35 | [18-50] | <0.001 |

Continuous data are shown as medians and interquartile ranges [Q1-Q3].
ECMO, extracorporeal membrane oxygenation; FFP, fresh frozen plasma; PGD, primary graft dysfunction; Plt, platelets; pRBC, packed red blood cells.

Predictors of PGD grade 3: Univariate logistic regression analysis of donor and recipient characteristics, and intra-operative outcomes, revealed age (Odds Ratio (OR) 0.97, 95% Confidence Interval (CI) 0.94-0.99, p<0.01), pre-transplant ECMO support (OR 9.33, 95% CI 4.04-21.6, p<0.001), Lung Allocation Score (OR 1.04, 95% CI 1.02-1.06, p<0.001), COVID-19 ARDS lung failure (OR 2.99, 95% CI 1.32-6.74, p<0.01), pre-operative hemoglobin (OR 0.82, 95% CI 0.72-0.93, p<0.01), pre-operative albumin (OR 0.29, 95% CI 0.15-0.56, p<0.001), pre-operative total bilirubin (OR 2.05, 95% CI 1.20-3.52, p<0.03), pre-operative INR (OR 6.05, 95% CI 1.18-31.1, p=0.03), post-reperfusion lung transplant biopsy C4d staining (OR 7.82, 95% CI 3.59-17.0, p<0.001), operative time (OR 1.39, 95% CI 1.16-1.67, p<0.001), intra-operative pRBC transfusion (OR 1.16, 95% CI 1.09-1.24, p<0.001), intra-operative FFP transfusion (OR 1.23, 95% CI 1.11-1.36, p<0.001), and intra-operative platelets transfusion (OR 1.54, 95% CI 1.24-1.90, p<0.001) as predictive of PGD grade 3 development (Table 3). On subsequent multivariate logistic regression analysis, post-reperfusion lung transplant biopsy C4d staining (OR 7.92, 95% CI 2.97-21.1, p<0.001) remained predictive (Table 4).

TABLE 3
Univariate logistic regression analysis to predict PGD grade 3.

| Variable | OR | 95% CI | P value |
|---|---|---|---|
| Recipient factors | | | |
| Age, years | 0.97 | 0.94-0.99 | <0.01 |
| Female | 1.42 | 0.70-2.91 | 0.34 |
| Body mass index, kg/m² | 0.97 | 0.90-1.05 | 0.51 |
| Body surface area, m² | 0.58 | 0.13-2.54 | 0.47 |
| Smoking history | 0.70 | 0.34-1.46 | 0.35 |
| Hypertension | 0.68 | 0.33-1.41 | 0.30 |
| Diabetes | 0.99 | 0.46-2.13 | 0.98 |
| Chronic Kidney Disease | 2.86 | 0.94-8.69 | 0.06 |
| Pre-transplant ECMO use | 9.33 | 4.04-21.6 | <0.001 |
| Bilateral lung transplant | 1.71 | 0.76-3.83 | 0.19 |
| Lung Allocation Score | 1.04 | 1.02-1.06 | <0.001 |
| Composite Allocation Score | 1.01 | 0.91-1.13 | 0.84 |
| Etiology of lung failure | | | |
| Interstitial Lung Disease | 0.82 | 0.39-1.73 | 0.60 |
| COVID-19 | 2.99 | 1.32-6.74 | <0.01 |
| COPD | 0.41 | 0.12-1.39 | 0.15 |
| Pulmonary Artery Hypertension | 0.60 | 0.13-2.69 | 0.50 |
| Other | 1.02 | 0.42-2.48 | 0.97 |
| Laboratory | | | |
| Hemoglobin, g/dL | 0.82 | 0.72-0.93 | <0.01 |
| WBC, 1,000/mm³ | 1.01 | 0.92-1.11 | 0.81 |
| Platelets, 1,000/mm³ | 1.00 | 0.99-1.00 | 0.05 |
| Sodium, mEq/L | 1.08 | 0.99-1.19 | 0.10 |
| BUN, mg/dL | 1.04 | 1.00-1.08 | 0.07 |
| Creatinine, mg/dL | 1.68 | 0.42-6.63 | 0.46 |
| AST, U/L | 1.01 | 1.00-1.03 | 0.10 |
| ALT, U/L | 1.00 | 0.98-1.02 | 0.78 |
| Albumin, g/dL | 0.29 | 0.15-0.56 | <0.001 |
| Total bilirubin, mg/dL | 2.05 | 1.20-3.52 | <0.01 |
| INR | 6.05 | 1.18-31.1 | 0.03 |
| PRA* | 1.93 | 0.94-3.96 | 0.07 |
| Arterial Blood Gas | | | |
| pH | 0.52 | 0.03-8.88 | 0.80 |
| PaCO₂ | 0.99 | 0.96-1.03 | 0.74 |
| PaO₂ | 1.00 | 0.99-1.00 | 0.05 |
| Donor | | | |
| Age, years | 1.00 | 0.97-1.03 | 0.81 |
| Female | 1.88 | 0.90-3.95 | 0.09 |
| Cause of death | | | |
| Anoxia | 0.93 | 0.44-1.93 | 0.84 |
| Head trauma | 1.11 | 0.53-2.29 | 0.79 |
| Other | 0.97 | 0.41-2.27 | 0.94 |
| Intra-operative outcomes | | | |
| Lung transplant biopsy C4d staining | 7.82 | 3.59-17.0 | <0.001 |
| Operative time (hours) | 1.39 | 1.16-1.67 | <0.001 |
| Intra-op blood transfusion; pRBC | 1.16 | 1.09-1.24 | <0.001 |
| Intra-op blood transfusion; FFP | 1.23 | 1.11-1.36 | <0.001 |
| Intra-op blood transfusion; Plt | 1.54 | 1.24-1.90 | <0.001 |
| Ischemic time (hours) | 0.89 | 0.69-1.15 | 0.38 |
| Veno-arterial ECMO use | 1.70 | 0.74-3.93 | 0.22 |
| Veno-arterial ECMO time (hours) | 1.08 | 0.88-1.32 | 0.48 |

ALT, Alanine aminotransferase; ARDS, acute respiratory distress syndrome; AST, aspartate aminotransferase; BUN, blood urea nitrogen; CI, confidence interval; COPD, chronic obstructive pulmonary disease; COVID-19, coronavirus disease 2019; ECMO, extracorporeal membrane oxygenation; FFP, fresh frozen plasma; INR, international normalized ratio; OR, Odds ratio; Plt, platelets; PGD, primary graft dysfunction; pRBC, packed red blood cells; WBC, white blood cell.
*PRA described as a count as this is an indicator of any sensitization (i.e. PRA > 0).

TABLE 4
Multivariate logistic regression analysis to predict PGD grade 3.

| Variable | OR | 95% CI | P value |
|---|---|---|---|
| Recipient factors | | | |
| Age, years | 1.00 | 0.97-1.04 | 0.86 |
| Pre-transplant ECMO use | 4.71 | 0.79-28.0 | 0.09 |
| Lung Allocation Score | 1.00 | 0.96-1.03 | 0.86 |
| Etiology of lung failure | | | |
| COVID-19 ARDS | 0.59 | 0.14-2.42 | 0.46 |
| Laboratory | | | |
| Hemoglobin, g/dL | 1.12 | 0.87-1.43 | 0.39 |
| Albumin, g/dL | 0.51 | 0.22-1.18 | 0.12 |
| Total bilirubin, mg/dL | 1.83 | 0.85-3.93 | 0.12 |
| Intra-operative outcomes | | | |
| Lung transplant biopsy C4d staining | 7.92 | 2.97-21.1 | <0.001 |
| Operative time (hours) | 1.19 | 0.89-1.73 | 0.32 |
| Intra-op blood transfusion; pRBC | 1.11 | 0.89-1.58 | 0.24 |
| Intra-op blood transfusion; FFP | 0.97 | 0.76-1.25 | 0.83 |
| Intra-op blood transfusion; Plt | 1.12 | 0.79-1.57 | 0.53 |

ARDS, Acute Respiratory Distress Syndrome; CI, confidence interval; COVID-19, Coronavirus 19; ECMO, extracorporeal membrane oxygenation; FFP, fresh frozen plasma; OR, Odds ratio; PGD, primary graft dysfunction; Plt, platelets; pRBC, packed red blood cells.

Patient characteristics and outcomes associated with post-reperfusion complement deposition: Forty-four (17.4%) patients had positive post-reperfusion lung transplant biopsy C4d staining. Patients with positive C4d staining were younger (53.9±13.4 vs. 59.3±12.0 years, p<0.01), female (26 (59.1%) vs. 78 (37.3%), p=0.01), with larger body surface area (1.8±0.3 vs. 1.9±0.2 m2, p=0.03), and had higher Lung Allocation Score (61.8±21.2 vs. 54.8±18.8, p=0.04) than those with negative C4d staining. Furthermore, patients with positive C4d staining had significantly lower pre-operative albumin (3.7±0.6 vs. 4.0±0.5 g/dL, p<0.01), and were more likely to have donors with anoxia as the cause of death (88 (42.1%) vs. 10 (22.7%), p=0.02). There were no other significant differences in recipient and donor characteristics between the two cohorts (Table 5).

TABLE 5
Patient Characteristics by post-reperfusion
lung transplant biopsy C4d staining.

| | C4d negative | | C4d positive | | |
|---|---|---|---|---|---|
| Variable | (N = 209) | | (N = 44) | | P value |
| Recipient factors | | | | | |
| Age, years | 59.3 ± 12.0 | | 53.9 ± 13.4 | | <0.01 |
| Female | 78 | (37.3%) | 26 | (59.1%) | 0.01 |
| Body Mass Index, kg/m² | 26.1 ± 4.5 | | 25.8 ± 5.0 | | 0.78 |
| Body Surface Area, m² | 1.9 ± 0.2 | | 1.8 ± 0.3 | | 0.03 |
| Smoking history | 102 | (48.8%) | 18 | (40.9%) | 0.41 |
| Hypertension | 112 | (53.6%) | 17 | (38.6%) | 0.10 |
| Diabetes | 71 | (34.0%) | 9 | (20.5%) | 0.11 |
| Chronic Kidney Disease | 14 | (6.7%) | 3 | (6.8%) | 1.00 |
| Pre-transplant ECMO use | 24 | (11.5%) | 8 | (18.2%) | 0.21 |
| Bilateral Lung Transplant | 134 | (64.1%) | 29 | (65.9%) | 0.86 |
| Lung Allocation Score | 54.8 ± 18.8 | | 61.8 ± 21.2 | | 0.04 |
| Composite Allocation Score | 26.8 ± 9.6 | | 26.1 ± 6.7 | | 0.86 |
| On the waiting list | 14 | [6-41] | 21 | [6-40] | 0.68 |
| Etiology of Lung Failure | | | | | |
| Interstitial Lung Disease | 83 | (39.7%) | 14 | (31.8%) | 0.40 |
| COVID-19 | 32 | (15.3%) | 8 | (18.2%) | 0.65 |
| COPD | 39 | (18.7%) | 5 | (11.4%) | 0.28 |
| Pulmonary Artery | 19 | (9.1%) | 3 | (6.8%) | 0.78 |
| Hypertension | | | | | |
| Other | 36 | (17.2%) | 14 | (31.8%) | 0.04 |
| Laboratory | | | | | |
| Hemoglobin, g/dL | 12.3 ± 2.4 | | 12.7 ± 1.5 | | 0.69 |
| WBC, 1,000/mm³ | 9.9 ± 3.3 | | 12.2 ± 3.6 | | 0.11 |
| Platelets, 1,000/mm³ | 244.9 ± 56.1 | | 275.1 ± 92.8 | | 0.27 |
| Sodium, mEq/L | 139.1 ± 3.8 | | 138.1 ± 5.1 | | 0.57 |
| BUN, mg/dL | 16.7 ± 6.3 | | 19.3 ± 8.5 | | 0.37 |
| Creatinine, mg/dL | 0.82 ± 0.22 | | 0.83 ± 0.22 | | 0.95 |
| AST, U/L | 25.9 ± 19.0 | | 29.0 ± 25.4 | | 0.36 |
| ALT, U/L | 21.4 ± 18.2 | | 20.3 ± 16.0 | | 0.70 |
| Albumin, g/dL | 4.0 ± 0.5 | | 3.7 ± 0.6 | | <0.01 |
| Total bilirubin, mg/dL | 0.6 ± 0.5 | | 0.7 ± 0.9 | | 0.26 |
| INR | 1.1 ± 0.2 | | 1.2 ± 0.4 | | 0.10 |
| PRA* | 83 | (39.7%) | 19 | (43.2%) | 0.74 |
| Arterial Blood Gas | | | | | |
| pH | 7.38 ± 0.07 | | 7.36 ± 0.08 | | 0.06 |
| PaCO2 | 48.8 ± 11.1 | | 50.5 ± 11.9 | | 0.38 |
| PaO2 | 266.2 ± 112.5 | | 266.3 ± 109.9 | | 1.00 |
| Donor | | | | | |
| Age, years | 33.9 ± 11.6 | | 30.8 ± 13.2 | | 0.11 |
| Female | 56 | (26.8%) | 15 | (34.1%) | 0.36 |
| Cause of Death | | | | | |
| Anoxia | 88 | (42.1%) | 10 | (22.7%) | 0.02 |
| Head trauma | 74 | (35.4%) | 22 | (50.0%) | 0.09 |
| Other | 47 | (22.5%) | 12 | (27.3%) | 0.56 |

Continuous data are shown as means ± standard deviation (SD) for age and laboratory data, and as medians and interquartile ranges [Q1-Q3] for days.
ALT, Alanine aminotransferase; ARDS, acute respiratory distress syndrome; AST, aspartate aminotransferase; BUN, blood urea nitrogen; COPD, chronic obstructive pulmonary disease; COVID-19, coronavirus disease 2019; ECMO; extracorporeal membrane oxygenation; INR, international normalized ratio; PGD, primary graft dysfunction; PRA, panel reactive antibodies; WBC, white blood cell.
Etiology of lung failure other: sarcoidosis, Hypersensitivity Pneumonitis, Cystic Fibrosis, Bronchiectasis, Obliterative bronchiolitis, Bronchoalveolar Carcinoma, Primary Ciliary Dyskinesia.
*PRA described as a count as this is an indicator of any sensitization (i.e. PRA > 0).

There were no significant differences in intra-operative outcomes between those patients with positive and negative C4d staining except for higher pRBC (2 [0-4] vs. 1 [0-3], p=0.04) and FFP transfusions (0 [0-2] vs. 0 [0-0], p=0.04) in the former. Post-operatively, patients with positive C4d staining were significantly more likely to develop PGD grade 3 (40.9% vs. 8.1%, p<0.001). Importantly, lower grades of complement deposition (grade 1 and 2) did not achieve statistical significance with development of PGD grade 3. In a cohort of patients who underwent double lung transplant Applicant isolated the bronchoalveolar lavage fluid from the new allografts between 2-4 hours following reperfusion after chest closure and found a high levels of correlation between grade of complement deposition in the tissue and soluble complement proteins (FIG. 4A). Additionally, Applicant found that single lung transplant recipients who had grade 3 complement staining at 30 minutes post-reperfusion had elevated levels of soluble C4d in the bronchoalveolar lavage obtained from the allograft but not from the native lung (FIG. 4B).

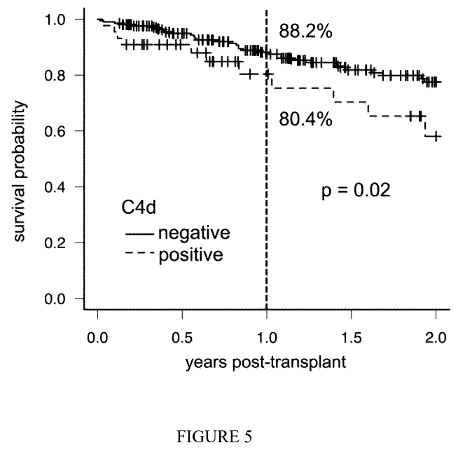

If used as a clinical test for PGD grade 3 in the study cohort, positive post-reperfusion lung transplant biopsy C4d staining had a sensitivity of 51.4%, specificity of 88.1%, positive predictive value of 40.9%, and negative predictive value of 86.1%. Additionally, positive C4d staining patients were more likely to develop digital ischemia (6.8% vs. 0.5%, p=0.02), and require dialysis (27.3% vs. 11.5%, p=0.02) and post-transplant ECMO support (25.0% vs. 12.4%, p=0.03) (Table 6). Notably, those patients with positive C4d staining had significantly decreased one-year survival compared to those with negative C4d staining (80.4% vs. 88.2%, p=0.02) (FIG. 5).

TABLE 6
Intra- and post-operative outcomes of lung transplant recipients
by post-reperfusion lung transplant biopsy C4d staining.

| | C4d negative | | C4d positive | | |
|---|---|---|---|---|---|
| Variable | (N = 209) | | (N = 44) | | P value |
| Intra-operative outcomes | | | | | |
| PGD 3 | 17 | (8.1%) | 18 | (40.9%) | <0.001 |
| Operative time (hours) | 6.2 | [5.0-8.0] | 6.2 | [5.1-8.1] | 0.79 |
| Intra-op blood transfusion; pRBC | 1 | [0-3] | 2 | [0-4] | 0.04 |
| Intra-op blood transfusion; FFP | 0 | [0-0] | 0 | [0-2] | 0.04 |
| Intra-op blood transfusion; Plt | 0 | [0-0] | 0 | [0-1] | 0.11 |
| Ischemic time (hours) | 5.2 | [4.2-5.8] | 5.2 | [4.3-6.5] | 0.31 |
| Veno-arterial ECMO use | 138 | (66.0%) | 34 | (77.3%) | 0.16 |
| Veno-arterial ECMO time (hours) | 2.4 | [0-3.1] | 2.5 | [0.2-3.3] | 0.26 |
| Post-operative outcomes | | | | | |
| AKI | 88 | (42.1%) | 24 | (54.5%) | 0.14 |
| Dialysis | 24 | (11.5%) | 12 | (27.3%) | 0.02 |
| Stroke | 4 | (1.9%) | 1 | (2.3%) | 1.00 |
| Bowel ischemia | 3 | (1.4%) | 1 | (2.3%) | 0.53 |
| Digital ischemia | 1 | (0.5%) | 3 | (6.8%) | 0.02 |
| Post-transplant ECMO use | 26 | (12.4%) | 11 | (25.0%) | 0.03 |
| Intensive Care Unit stay (days) | 8 | [5-18] | 9 | [6-21] | 0.45 |
| Post-transplant ventilator (days) | 2 | [1-3] | 2 | [1-9] | 0.25 |
| Hospital stay (days) | 18 | [12-29] | 19 | [13-36] | 0.41 |

Continuous data are shown as medians and interquartile ranges [Q1-Q3].
ECMO, extracorporeal membrane oxygenation; FFP, fresh frozen plasma; PGD, primary graft dysfunction; Plt, platelets; pRBC, packed red blood cells.

### Discussion

The utilization of lung transplantation as a therapeutic strategy for both acute and chronic end-stage lung diseases has increased over time. However, its outcomes lag other solid organ transplants, partially due to the high incidence of PGD.^{2-7, 33-37} This study is the first to investigate the correlation between post-reperfusion lung transplant biopsy C4d staining and PGD, thereby assessing its predictive value for significant PGD development. Results from this study demonstrate that positive post-reperfusion C4d staining is associated with severe PGD, exhibiting high specificity in predicting PGD 3 at 72 hours. Early identification of lung transplant recipients predisposed to severe PGD, might enable earlier interventions.

This study aimed to analyze post-reperfusion lung transplant biopsy C4d staining as it provides an opportunity for earlier detection compared to previously studied post-operative complement parameters by using an intraoperative lung allograft biopsy, a simple and safe intervention performed at the time of transplant which was not associated with any adverse events in the study. Furthermore, being a wedge biopsy of lung tissue, it offers a more holistic assessment of complement activation within the tissue and correlation with histological evidence of tissue damage.

There was a significantly higher occurrence of positive post-reperfusion C4d staining in patients with PGD grade 3 compared to those with PGD grades 0-2. Additionally, when stratifying patients based on C4d staining, those with higher grades of positive post-reperfusion C4d staining were significantly more likely to experience PGD grade 3 than those with negative or lower grades of C4d staining.

Notably, patients with PGD grade 3 were found to require longer operative times and more intra-operative blood products. These factors potentially suggest that patients with more severe disease prior to transplant might be more susceptible to the activation of complement, consistent with prior reports.³⁹

While multiple recipient characteristics and intra-operative outcomes were found to be predictive on the univariate analysis (Table 3), only C4d staining remained a significant predictor on the multivariate analysis (Table 4). Together, these results support the use of positive post-reperfusion lung transplant biopsy C4d staining as an early clinical marker of PGD.

While the data shows C4d staining is not a sensitive test for significant PGD, it did have high specificity, in addition to a high negative predictive value. These data, particularly the low sensitivity of C4d in predicting PGD, underscores the multifactorial etiology of PGD with ischemia-reperfusion remaining the predominant factor.

Without wishing to be bound by any theory, early deposition of complement could potentially be used to evaluate the role of future therapies such as complement inhibition to ameliorate PGD severity at 72 hours and enhance clinical outcomes. Prior efforts to use complement inhibition to mitigate PGD have yielded mixed results likely due to the non-selective treatment of all recipients in the intervention arm. However, treatment directed at inhibiting complement in those patients that demonstrate tissue complement deposition could be more efficacious.

Furthermore, the complement deposition can be detected using both immunofluorescence and immunohistochemistry. Here, Applicant used immunohistochemistry. Despite immunohistochemistry requiring a longer time than immunofluorescence, it can result within the first 24 hours. Applicant has found complete harmony between immunofluorescence which can be tested within a few hours and immunohistochemistry which takes about 24 hours (data not shown), but further research is needed to validate this as current literature shows some discordance between immunofluorescence and immunohistochemistry C4d staining.^{26, 52, 53} Since PGD at 72 hours is the most important predictor for long-term outcomes, this might enable future studies to implement therapies between days 1-3 to mitigate PGD risk. Alternatively, soluble complement in bronchoalveolar lavage can be used as a surrogate for tissue complement as Applicant found a high correlation between the two, however the allograft biopsy is preferred as it shows tissue damage and complement protein deposition in the tissue. Complement protein in lung fluid may or may not indicate a need for additional therapy.

Therefore, assessment of tissue complement deposition combined with HLA and autoantibody assessment, as described in a recent report,¹⁷ can guide antibody and complement directed therapies for PGD.

### Conclusion

This study demonstrated a significant association between PGD grade 3 at 72 hours and positive post-reperfusion lung transplant biopsy C4d staining. Furthermore, Applicant found that positive C4d staining is a reliable predictor of severe PGD. This relationship was uncovered via safe intraoperative lung biopsy procedures.

### Embodiments

Exemplary embodiments include, but are not limited to:

1. A method for predicting primary graft dysfunction in a subject comprising quantifying a complement protein in an allograft biopsy, wherein the allograft biopsy is obtained from an organ up to 120 minutes post-reperfusion following the transplant of the organ in the subject, and wherein the complement protein in the allograft biopsy is at least 1.2-fold greater than a quantity of the same complement protein in a biopsy from the same organ at the end of cold ischemia.

2. The method of embodiment 1, wherein the complement protein is not at quantifiable levels in the biopsy from the organ at the end of cold ischemia.

3. The method of embodiment 1, wherein the allograft biopsy is obtained at 30 minutes post-reperfusion.

4. The method of embodiment 1, wherein the allograft biopsy is obtained between 30 and 120 minutes post-reperfusion.

5. The method of embodiment 1, wherein the allograft biopsy is obtained up to 90 minutes post-reperfusion.

6. The method of embodiment 1, wherein the complement protein is C4d.

7. The method of embodiment 1, wherein the complement protein is selected from a group comprising C3d, C5b-9, and C4d.

8. The method of embodiment 1, wherein the quantifying of a complement protein is done with immunohistochemical staining.

9. The method of embodiment 1, wherein the quantifying of a complement protein is done with immunofluorescence.

10. The method of embodiment 1, wherein the biopsies are wedge biopsies.

11. The method of embodiment 1, wherein the method predicts severe primary graft dysfunction.

12. The method of embodiment 1, wherein the method predicts mild primary graft dysfunction.

13. The method of embodiment 1, wherein the quantity of the complement protein in the allograft biopsy can further be used to predict post-transplant outcomes, selected from the group comprising digital ischemia, post-transplant Extracorporeal Membrane Oxygenation (ECMO) support, and a need for dialysis.

14. The method of embodiment 1, wherein the detection and/or presence of the complement protein in the allograft biopsy can also be used to predict post-transplant outcomes, selected from a group comprising digital ischemia, post-transplant ECMO support, and a need for dialysis.

15. The method of embodiment 1, wherein the organ is a lung.

16. The method of embodiment 1, wherein the organ is a heart.

17. A method for predicting primary graft dysfunction in a subject comprising detecting a complement protein in an allograft biopsy, wherein the allograft biopsy is obtained from an organ up to 120 minutes post-reperfusion following the transplant of the organ in the subject.

18. The method of embodiment 17, wherein the detecting of a complement protein is done with immunohistochemical staining.

19. The method of embodiment 17, wherein the detecting of a complement protein is done with immunofluorescence.

20. The method of embodiment 17, wherein the complement protein is C4d.

21. The method of embodiment 17, wherein the complement protein is selected from a group comprising C3d, C5b-9, and C4d.

22. The method of embodiment 17, wherein the organ is a lung.

23. The method of embodiment 17, wherein the organ is a heart.

24. A method for treating primary graft dysfunction in a subject comprising detecting a complement protein in an allograft biopsy, wherein the allograft biopsy is obtained from an organ up to 120 minutes post-reperfusion following the transplant of the organ in the subject, and treating the subject with a treatment for primary graft dysfunction when the complement protein level is at least 1.2-fold higher than a quantity of the same complement protein in a biopsy from the same organ at the end of cold ischemia.

25. The method of embodiment 24, wherein the treatment for primary graft dysfunction is complement inhibition therapy.

26. A method for predicting primary graft dysfunction in a subject comprising detecting a complement protein in a transbronchial biopsy, wherein the transbronchial biopsy is obtained from a lung up to 72 hours post-transplant of the lung in the subject.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above.

All numerical designations, e.g., temperature, time, and amounts, including ranges, are approximations which are varied (+) or (−) by 10%, 1%, or 0.1%, as appropriate. It is to be understood, although not always explicitly stated, that all numerical designations may be preceded by the term “about.”

All patents, patent applications, provisional applications, and publications referred to or cited herein are incorporated by reference in their entirety, including all figures and tables, to the extent they are not inconsistent with the explicit teachings of this specification.

### REFERENCES

1. Snell G I, Yusen R D, Weill D, et al. Report of the ISHLT Working Group on Primary Lung Graft Dysfunction, part I: Definition and grading—A 2016 Consensus Group statement of the International Society for Heart and Lung Transplantation. The Journal of heart and lung transplantation: the official publication of the International Society for Heart Transplantation. October 2017; 36(10):1097-1103. doi:10.1016/j.healun.2017.07.021
2. Samano M N, Fernandes L M, Baranauskas J C B, et al. Risk Factors and Survival Impact of Primary Graft Dysfunction After Lung Transplantation in a Single Institution. Transplantation Proceedings. 2012 Oct. 1 2012; 44(8):2462-2468. doi:10.1016/j.transproceed.2012.07.134
3. Wilkey B J, Abrams B A. Mitigation of Primary Graft Dysfunction in Lung Transplantation: Current Understanding and Hopes for the Future. Seminars in Cardiothoracic and Vascular Anesthesia. 2020; 24(1):54-66. doi:10.1177/1089253219881980
4. Daud S A, Yusen R D, Meyers B F, et al. Impact of Immediate Primary Lung Allograft Dysfunction on Bronchiolitis Obliterans Syndrome. American Journal of Respiratory and Critical Care Medicine. 2007; 175(5):507-513. doi:10.1164/rccm.200608-1079OC
5. Christie J D, Kotloff R M, Ahya V N, et al. The effect of primary graft dysfunction on survival after lung transplantation. Am J Respir Crit Care Med. Jun. 1 2005; 171(11):1312-6. doi:10.1164/rccm.200409-1243OC
6. Diamond J M, Arcasoy S, Kennedy C C, et al. Report of the International Society for Heart and Lung Transplantation Working Group on Primary Lung Graft Dysfunction, part II: Epidemiology, risk factors, and outcomes-A 2016 Consensus Group statement of the International Society for Heart and Lung Transplantation. J Heart Lung Transplant. October 2017; 36(10):1104-1113. doi:10.1016/j.healun.2017.07.020
7. Whitson B A, Nath D S, Johnson A C, et al. Risk factors for primary graft dysfunction after lung transplantation. The Journal of Thoracic and Cardiovascular Surgery. 2006 Jan. 1/2006; 131(1):73-80. doi: 10.1016/j.jtcvs.2005.08.039
8. Shaver C M, Ware L B. Primary graft dysfunction: pathophysiology to guide new preventive therapies. Expert Rev Respir Med. February 2017; 11(2):119-128. doi:10.1080/17476348.2017.1280398
9. Gelman A E, Fisher A J, Huang H J, et al. Report of the ISHLT Working Group on Primary Lung Graft Dysfunction Part III: Mechanisms: A 2016 Consensus Group Statement of the International Society for Heart and Lung Transplantation. J Heart Lung Transplant. October 2017; 36(10):1114-1120. doi:10.1016/j.healun.2017.07.014
10. Bishop M J, Giclas P C, Guidotti S M, Su M L, Chi E Y. Complement activation is a secondary rather than a causative factor in rabbit pulmonary artery ischemia/reperfusion injury. Am Rev Respir Dis. February 1991; 143(2):386-90. doi:10.1164/ajrccm/143.2.386
11. Naka Y, Marsh H C, Scesney S M, Oz M C, Pinsky D J. Complement activation as a cause for primary graft failure in an isogeneic rat model of hypothermic lung preservation and transplantation. Transplantation. Nov. 15 1997; 64(9):1248-55. doi:10.1097/00007890-199711150-00004
12. Ali H A, Pavlisko E N, Snyder L D, Frank M, Palmer S M. Complement system in lung transplantation. Clin Transplant. April 2018; 32(4):e13208. doi:10.1111/ctr.13208
13. Zheng Z, Chiu S, Akbarpour M, et al. Donor pulmonary intravascular nonclassical monocytes recruit recipient neutrophils and mediate primary lung allograft dysfunction. Sci Transl Med. Jun. 14 2017; 9(394)doi:10.1126/scitranslmed.aal4508
14. Kurihara C, Lecuona E, Wu Q, et al. Crosstalk between nonclassical monocytes and alveolar macrophages mediates transplant ischemia-reperfusion injury through classical monocyte recruitment. JCI Insight. Mar. 22 2021; 6(6)doi:10.1172/jci.insight.147282
15. Hsiao H M, Fernandez R, Tanaka S, et al. Spleen-derived classical monocytes mediate lung ischemia-reperfusion injury through IL-1beta. J Clin Invest. Jul. 2 2018; 128(7):2833-2847. doi:10.1172/JCI98436
16. Camous L, Roumenina L, Bigot S, et al. Complement alternative pathway acts as a positive feedback amplification of neutrophil activation. Blood. Jan. 27 2011; 117(4):1340-9. doi:10.1182/blood-2010-05-283564
17. Yang W, Cerier E J, Núñez-Santana F L, et al. IL-1β-dependent extravasation of preexisting lung-restricted autoantibodies during lung transplantation activates complement and mediates primary graft dysfunction. J Clin Invest. Oct. 17 2022; 132(20)doi:10.1172/jci157975
18. Toyoda T C E, Manerikar A, et al. Recipient, donor and surgical factors leading to primary graft dysfunction after lung transplant. Journal of Thoracic Disease. 2023;
19. Westall G P, Snell G I, McLean C, Kotsimbos T, Williams T, Magro C. C3d and C4d deposition early after lung transplantation. J Heart Lung Transplant. July 2008; 27(7):722-8. doi:10.1016/j.healun.2008.03.018
20. Shah R J, Emtiazjoo A M, Diamond J M, et al. Plasma complement levels are associated with primary graft dysfunction and mortality after lung transplantation. Am J Respir Crit Care Med. Jun. 15 2014; 189(12):1564-7. doi:10.1164/rccm.201312-2121LE
21. Keshavjee S, Davis R D, Zamora M R, de Perrot M, Patterson G A. A randomized, placebo-controlled trial of complement inhibition in ischemia-reperfusion injury after lung transplantation in human beings. J Thorac Cardiovasc Surg. February 2005; 129(2):423-8. doi:10.1016/j.jtcvs.2004.06.048
22. Goetz R, Houp J, Rusanov V, et al. ECULIZUMAB A S SALVAGE THERAPY FOR LUNG TRANSPLANT IN A SENSITIZED PATIENT WITH A POSITIVE T AND B CELL CROSS MATCH AND SEVERE ALLOGRAFT DYSFUNCTION. CHEST. 2021; 160(4):A2474. doi:10.1016/j.chest.2021.07.2139
23. Cantu E, Diamond J M, Cevasco M, et al. Contemporary trends in PGD incidence, outcomes, and therapies. The Journal of heart and lung transplantation: the official publication of the International Society for Heart Transplantation. December 2022; 41(12):1839-1849. doi:10.1016/j.healun.2022.08.013
24. Diamond J M, Lee J C, Kawut S M, et al. Clinical risk factors for primary graft dysfunction after lung transplantation. Am J Respir Crit Care Med. Mar. 1 2013; 187(5):527-34. doi:10.1164/rccm.201210-1865OC
25. Copeland H, Hayanga J W A, Neyrinck A, et al. Donor heart and lung procurement: A consensus statement. The Journal of heart and lung transplantation: the official publication of the International Society for Heart Transplantation. June 2020; 39(6):501-517. doi:10.1016/j.healun.2020.03.020
26. Roden A C, Maleszewski J J, Yi E S, et al. Reproducibility of Complement 4d deposition by immunofluorescence and immunohistochemistry in lung allograft biopsies. J Heart Lung Transplant. December 2014; 33(12):1223-32. doi:10.1016/j.healun.2014.06.006
27. Berry G J, Burke M M, Andersen C, et al. The 2013 International Society for Heart and Lung Transplantation Working Formulation for the standardization of nomenclature in the pathologic diagnosis of antibody-mediated rejection in heart transplantation. J Heart Lung Transplant. December 2013; 32(12):1147-62. doi:10.1016/j.healun.2013.08.011
28. Toyoda T, Thomae B L, Kandula V, et al. Primary graft dysfunction grade correlates with acute kidney injury stage after lung transplantation. J Thorac Dis. Jul. 31 2023; 15(7):3751-3763. doi:10.21037/jtd-23-256
29. Cerier E, Manerikar A, Kandula V, et al. Post-reperfusion pulmonary artery pressure indicates primary graft dysfunction after lung transplant. Ann Thorac Surg. Dec. 12 2022; doi:10.1016/j.athoracsur.2022.12.013
30. Kurihara C, Manerikar A, Gao C A, et al. Outcomes after extracorporeal membrane oxygenation support in COVID-19 and non-COVID-19 patients. Artif Organs. April 2022; 46(4):688-696. doi:10.1111/aor.14090
31. Manerikar A, Watanabe S, Kandula V, et al. Indwelling Central Venous Catheters Drive Bloodstream Infection During Veno-venous Extracorporeal Membrane Oxygenation Support. Asaio j. Jun. 1 2022; 68(6):859-864. doi:10.1097/mat.0000000000001575
32. Kurihara C, Walter J M, Karim A, et al. Feasibility of Venovenous Extracorporcal Membrane Oxygenation Without Systemic Anticoagulation. Ann Thorac Surg. October 2020; 110(4):1209-1215. doi:10.1016/j.athoracsur.2020.02.011
33. van der Mark S C, Hock R A S, Hellemons M E. Developments in lung transplantation over the past decade. Eur Respir Rev. Sep. 30 2020; 29(157)doi:10.1183/16000617.0132-2019
34. Chambers D C, Cherikh W S, Harhay M O, et al. The International Thoracic Organ Transplant Registry of the International Society for Heart and Lung Transplantation: Thirty-sixth adult lung and heart-lung transplantation Report-2019; Focus theme: Donor and recipient size match. J Heart Lung Transplant. October 2019; 38(10):1042-1055. doi:10.1016/j.healun.2019.08.001
35. Kurihara C, Manerikar A, Querrey M, et al. Clinical Characteristics and Outcomes of Patients With COVID-19—Associated Acute Respiratory Distress Syndrome Who Underwent Lung Transplant. JAMA. 2022; 327(7):652-661. doi:10.1001/jama.2022.0204
36. Bos S, Vos R, Van Raemdonck D E, Verleden G M. Survival in adult lung transplantation: where are we in 2020? Curr Opin Organ Transplant. June 2020; 25(3):268-273. doi:10.1097/mot.0000000000000753
37. Israni A K. OPTN/SRTR 2020 Annual Data Report: Introduction. American Journal of Transplantation. 2022; 22(S2):11-20. doi:10.1111/ajt. 16974
38. Kulkarni H S, Ramphal K, Ma L, et al. Local complement activation is associated with primary graft dysfunction after lung transplantation. JCI Insight. Sep. 3, 2020; 5(17)doi: 10.1172/jci.insight.138358
39. Bharat A, Saini D, Steward N, et al. Antibodies to self-antigens predispose to primary lung allograft dysfunction and chronic rejection. The Annals of thoracic surgery. October 2010; 90(4):1094-101. doi:10.1016/j.athoracsur.2010.06.009
40. Pierre A F, Xavier A M, Liu M, et al. Effect of complement inhibition with soluble complement receptor 1 on pig allotransplant lung function. Transplantation. Sep. 27 1998; 66(6):723-32. doi:10.1097/00007890-199809270-00006
41. Schmid R A, Zollinger A, Singer T, et al. Effect of soluble complement receptor type 1 on reperfusion edema and neutrophil migration after lung allotransplantation in swine. J Thorac Cardiovasc Surg. July 1998; 116(1):90-7. doi:10.1016/s0022-5223(98)70246-6
42. Scherer M, Demertzis S, Langer F, Moritz A, Schäfers H J. C1-esterase inhibitor reduces reperfusion injury after lung transplantation. Ann Thorac Surg. January 2002; 73(1):233-8; discussion 238-9. doi:10.1016/s0003-4975(01)03235-0
43. Stammberger U, Hamacher J, Pache J C, Schmid R A. sCR1sLe(X) reduces lung allograft ischemia-reperfusion injury but does not ameliorate acute rejection. Eur J Cardiothorac Surg. September 2002; 22(3):368-72. doi:10.1016/s1010-7940(02)00355-x
44. Stammberger U, Hamacher J, Hillinger S, Schmid R A. sCR1sLe ameliorates ischemia/reperfusion injury in experimental lung transplantation. J Thorac Cardiovasc Surg. December 2000; 120(6):1078-84. doi:10.1067/mtc.2000.111175
45. Salvatierra A, Velasco F, Rodriguez M, et al. C1-esterase inhibitor prevents early pulmonary dysfunction after lung transplantation in the dog. Am J Respir Crit Care Med. March 1997; 155(3):1147-54. doi:10.1164/ajrccm.155.3.9117001
46. Sommer W, Tudorache I, Kühn C, et al. C1-esterase-inhibitor for primary graft dysfunction in lung transplantation. Transplantation. Jun. 15 2014; 97(11):1185-91. doi:10.1097/tp.0000000000000034
47. Li W, Terada Y, Tyurina Y Y, et al. Necroptosis triggers spatially restricted neutrophil-mediated vascular damage during lung ischemia reperfusion injury. Proc Natl Acad Sci USA. Mar. 8 2022; 119(10):e2111537119. doi:10.1073/pnas.2111537119
48. Pottecher J, Roche A C, Dégot T, et al. Increased Extravascular Lung Water and Plasma Biomarkers of Acute Lung Injury Precede Oxygenation Impairment in Primary Graft Dysfunction After Lung Transplantation. Transplantation. January 2017; 101(1):112-121. doi:10.1097/tp.0000000000001434
49. Chang D H, Kittleson M, Patel J, et al. (373)—Treatment of Primary Graft Dysfunction with Plasmapheresis Improves Outcome. The Journal of Heart and Lung Transplantation. 2017 Apr. 1/2017; 36(4, Supplement):S147. doi:10.1016/j.healun.2017.01.385
50. Howard M C, Nauser C L, Farrar C A, Sacks S H. Complement in ischaemia-reperfusion injury and transplantation. Semin Immunopathol. December 2021; 43(6):789-797. doi:10.1007/s00281-021-00896-3
51. Park C S, Hwang S, Park H W, et al. Role of Plasmapheresis as Liver Support for Early Graft Dysfunction Following Adult Living Donor Liver Transplantation. Transplantation Proceedings. 2012 Apr. 1/2012; 44(3):749-751. doi:10.1016/j.transproceed.2012.01.054
52. Kalantari Tannenbaum S, Uraizee I, Grinstein J, et al. Discordance between C4d Staining by Immunohistochemistry and Immunofluorescence and Clinical Outcomes. The Journal of Heart and Lung Transplantation. 2016; 35(4):S40-S41. doi:10.1016/j.healun.2016.01.109
53. Fujino T, Kumai Y, Yang B, et al. Discordance between immunofluorescence and immunohistochemistry C4d staining and outcomes following heart transplantation. Clin Transplant. April 2021; 35(4):e14242. doi:10.1111/ctr.14242
54. Hull, T. D. et al., Primary graft dysfunction in heart transplantation: How to recognize it, when to institute extracorporeal membrane oxygenation, and outcomes. JCTVS Open. 2021; 8:128-133. 10.1016/j.xjon.2021.05.010
55. Giangreco N, et al., Abstract 19311: Identification of Novel Primary Graft Dysfunction Biomarkers Using Exosome Proteomics. Circulation. 2018; 136:A19311.

What is claimed is:

1. A method for predicting primary graft dysfunction in a subject comprising quantifying a complement protein in an allograft biopsy, wherein the allograft biopsy is obtained from an organ up to 120 minutes post-reperfusion following the transplant of the organ in the subject, and wherein the complement protein in the allograft biopsy is at least 1.2-fold greater than a quantity of the same complement protein in a biopsy from the same organ at the end of cold ischemia.

2. The method of claim 1, wherein the complement protein is not at quantifiable levels in the biopsy from the organ at the end of cold ischemia.

3. The method of claim 1, wherein the allograft biopsy is obtained at 30 minutes post-reperfusion.

4. The method of claim 1, wherein the allograft biopsy is obtained between 30 and 120 minutes post-reperfusion.

5. The method of claim 1, wherein the allograft biopsy is obtained up to 90 minutes post-reperfusion.

6. The method of claim 1, wherein the complement protein is C4d.

7. The method of claim 1, wherein the complement protein is selected from a group comprising C3d, C5b-9, and C4d.

8. The method of claim 1, wherein the quantifying of a complement protein is done with immunohistochemical staining or immunofluorescence.

9. The method of claim 1, wherein the biopsies are wedge biopsies.

10. The method of claim 1, wherein the method predicts severe primary graft dysfunction or mild primary graft dysfunction.

11. The method of claim 1, wherein the quantity of the complement protein in the allograft biopsy can further be used to predict post-transplant outcomes, selected from the group comprising digital ischemia, post-transplant Extracorporeal Membrane Oxygenation (ECMO) support, and a need for dialysis.

12. The method of claim 1, wherein the presence of the complement protein in the allograft biopsy can be used to predict post-transplant outcomes, selected from a group comprising digital ischemia, post-transplant ECMO support, and a need for dialysis.

13. The method of claim 1, wherein the organ is a lung or a heart.

14. A method for predicting primary graft dysfunction in a subject comprising detecting a complement protein in an allograft biopsy, wherein the allograft biopsy is obtained from an organ up to 120 minutes post-reperfusion following the transplant of the organ in the subject.

15. The method of claim 14, wherein the detecting of a complement protein is done with immunohistochemical staining or immunofluorescence.

16. The method of claim 14, wherein the complement protein is C4d.

17. The method of claim 14, wherein the complement protein is selected from a group comprising C3d, C5b-9, and C4d.

18. The method of claim 14, wherein the organ is a lung or heart.

19. A method for treating primary graft dysfunction in a subject comprising detecting a complement protein in an allograft biopsy, wherein the allograft biopsy is obtained from an organ up to 120 minutes post-reperfusion following the transplant of the organ in the subject, and treating the subject with a treatment for primary graft dysfunction when the complement protein level is at least 1.2-fold higher than a quantity of the same complement protein in a biopsy from the same organ at the end of cold ischemia.

20. The method of claim 19, wherein the treatment for primary graft dysfunction is complement inhibition therapy.
